(19) United States
(12) Patent Application Publication
(10) Pub. No.: US 2006/0235530 A1
(43) Pub. Date: Oct. 19, 2006

(54) Artificial prosthesis

(76) Inventor: Alexis Paul Shelokov, Dallas, TX (US)

Correspondence Address: CHALKER FLORES, LLP, 2711 LBJ FRWY, Suite 1036, DALLAS, TX, 75234, US

(21) Appl. No.: 11/316,289

(22) Filed: Dec. 22, 2005

Related U.S. Application Data
(60) Provisional application No. 60/672,318, filed on Apr. 18, 2005.

(51) Int. Cl. A61F 2/44; A61F 2/30

(52) U.S. Cl. 623/17.15; 623/23.39

(57) ABSTRACT

The present invention is directed to an artificial prosthesis (50) that includes a first disc (56) with a first disc periphery (60), a first disc superior surface (58) and a first disc inferior surface (68) having a first mating portion (90), a second disc (54) having a second mating portion (80) that is substantially complementary to the first disc mating portion (90) and a convex surface (65), and a third disc (52) having a concave superior surface (62) that substantially complements the convex surface of the second disc (65).

### CROSS-REFERENCE TO RELATED APPLICATIONS

This application claims priority to U.S. Provisional Application Ser. No. 60/672,318, filed Apr. 18, 2005, the contents of which are incorporated by reference herein in its entirety.

### TECHNICAL FIELD OF THE INVENTION

The present invention relates in general to the field of artificial prosthesis, and more particularly, to an improved artificial prosthesis for use in various joints with reduced material fatigue and improved patient comfort.

### BACKGROUND OF THE INVENTION

Without limiting the scope of the invention, its background is described in connection with spinal joint prosthesis.

A wide variety of artificial prosthesis are used to replace chronically injured or ruptured intervertebral discs; primarily, in the lower lumbar region of the spine. These prostheses generally employ one, two, three or more individual elements having a wide range of constructs including ball and socket joints, gel filled enclosures, spring-biased plates, plate and joint combinations and others. Prosthetic spinal discs have been used and developed primarily for the lumbar spine.

For example, U.S. Pat. No. 6,039,763, issued to the present inventor, discloses and claims an articulating spinal disc prosthesis that is designed to articulate in a manner resembling a human knee articulation. Briefly, the artificial spinal disc includes two plates each having a planar outer surface and an articulating inner surface. The planar outer surfaces contact and are affixed to adjacent inferior and superior surfaces of two adjacent vertebrae. The interior articulating surfaces of the artificial spinal disc are generally shaped to have one or more complementary convex/concave shaped articulating portions that are shaped and articulate in a manner similar to a human knee.

Another example of an intervertebral disc prosthesis is taught in U.S. Pat. No. 5,401,269, issued to Buttner-Janz, et al. These inventors disclose an intervertebral disc endoprosthesis that includes two prosthesis plates that have a prosthesis core which cooperates with at least one prosthesis plate via an articulating surface permitting a rotational movement. The two plates are connected to the end plates of adjacent vertebrae. To control rotational movement around the vertical axis, the articulating surface forms curved arches of different average radius in the median section and in the frontal section. The radius of curvature in the sagittal section is less than in the frontal section.

More recently, U.S. Pat. No. 6,846,328, issued to Cauthen teaches another articulating spinal implant. The articulating intervertebral disc replacement implant is formed from two elements, each engaging one of an adjacent pair of vertebra and capable of an articulating motion that resists compression and lateral movement between the vertebra, but allows the adjacent vertebra to articulate about an instantaneous axis of rotation.

### SUMMARY OF THE INVENTION

The present inventor has recognized that most interosseal prosthetic devices suffer from disadvantages such as the movement of the end plates, dislocation of the endplates, cold flow, ossification of the annulus, and particulate wear and debris of the component parts. There remains a need for artificial implants, e.g., artificial intervertebral disc prosthesis that mimic natural spinal segmental motion and provides an improved alternative to spinal fusion. Similar problems have been found with interosseal implants for use with a wide variety of joints, e.g., degenerative hips and knees. The present invention was developed to address these and other limitations of existing device, e.g., the “battery” effect created on apposed surfaces cause by the inherent induction of capacitance created across joints and prosthesis that lead to damage of the implant and, potentially, device failure. The present invention also takes into consideration the compressive forces and increased potential for fractures at the implant-joint interface, e.g., the load-bearing rim portion of the vertebrae.

The present invention finds widespread use in all interosseal implant locations by providing a viable mechanical substitute with enhance function, decreased pain and the durability necessary for long-term use. For example, when used in conjunction with bone morphogenic protein, the present invention leads to a reduction in recovery time, improved natural segmental motion, pain relief and durability for long-term use.

More particularly, the present invention is a three part or disc artificial prosthesis. For example, an intervertebral implant may include a first disc having a first disc periphery, a first disc superior surface and a first disc inferior surface with a first disc mating portion; a second disc with a second disc periphery, a second disc superior surface having a second disc mating portion that is substantially complementary to the first disc mating portion and a convex inferior surface. The third disc includes a concave superior surface that substantially complements the convex inferior surface of the second disc. In operation, the first, the second or both mating portions may further include one or more grooves that extend to the first, second or both outer disc periphery, wherein the one or more grooves are in fluid communication at a site of implantation that permit ion transfer at, through and about the outer periphery of the first and second discs. In addition to ion flow, these one or more grooves may be shaped to create a mechanical fluid pump that causes the movement of fluid through the grooves. Additionally, the one or more grooves may also form one or more reservoirs adapted to collect particulate matter, thereby reducing the potential for friction between plates and increases device longevity. The one or more reservoirs may further include one or more active agents, e.g., one or more growth factors, one or more bone morphogenic protein isoforms, steroids, collagen, glucocorticoids, analgesics, anti-inflammatories and mixtures and combinations. The reservoir may be accessible externally such that matter may be removed (e.g., debris) or inserted (e.g., to change or replace the active agents).

The first, second or third disc may be made from stainless steel, titanium, cobalt, aluminum, polymer(s), ceramics, carbon fiber, alloys and composites materials or combinations thereof. The materials may be used alone or in combination, e.g., a disc may have a core made from one material (e.g., a biocompatible polymer) and a periphery that includes metal and ceramic.

When used as an intervertebral prosthesis, the center of gravity of the convex surface of the third disc may be off-center. In one specific embodiment, the first and second mating portions may be limited to a range of motion of about 17 degrees from center. The exact location of the off-center center of gravity of the convex surface of the third disc may be selected and varied to take into account the exact location of the implant to compensate for the curvature of the spine. In some cases, the location of the center of gravity for the convex surface may be anterior, posterior, medial or lateral. As such, the center of gravity may even be customized to address vertebral disorder or discomfort caused by, e.g., kyphosis, lordosis or scoliosis.

When used in any joint, apex of the convex portion of the third disc may be selected to control or improve lateral flexion, rotation or flexion/extension depending on the site of implantation. Furthermore, the discs may also include an inferior surface comprising a periphery that complements and attaches to the load-bearing rim of a bone upon implantation. Depending on the site of implantation, the prosthesis may be an artificial shoulder joint, an artificial hip joint, an artificial knee joint, an artificial elbow joint, an artificial ankle joint, an artificial wrist joint, an artificial carpo-metacarpal joint, an artificial metacarpo-phalangeal joint, an artificial interphalangeal joint and an artificial metatarso-phalangeal joint, to list a few. To provide additional mechanical strength to the prosthesis upon implantation, the first, the third or both discs may include one or more holes therethrough for use in securing the first, the third or both discs to a bone. The first, the second, the third outer disk periphery, and combinations thereof, may be chamfered, kidney shaped and/or coated.

The present invention also includes a method for replacing a joint in a human including the steps of removing a joint from a human to provide an space defined by a first and a second bone; inserting an prosthesis as disclosed herein within the space; and attaching the prosthesis to the first and second bones. The prosthesis may be intervertebral, a shoulder, a hip, a knee, an elbow, an ankle, a wrist, a carpo-metacarpal, a metacarpo-phalangeal, an interphalangeal and a metatarso-phalangeal. The surface of each first and second bone may be adapted to be substantially complementary to the superior of the first disc and inferior surface of the third disc. Alternatively, the outer periphery of the first and third disc may be adapted to be substantially complementary to the bone at the site of implantation or both may be adapted.

Yet another method of the present invention replaces an intervertebral joint in a patient in need thereof by removing a spinal disc from the spine to provide an intervertebral space defined by a superior vertebra and an inferior vertebra; inserting an the prosthesis of the present invention in the intervertebral space; and attaching the prosthesis to the superior and inferior vertebrae.

### BRIEF DESCRIPTION OF THE DRAWINGS

For a more complete understanding of the features and advantages of the present invention, reference is now made to the detailed description of the invention along with the accompanying figures and in which:

FIG. 1 is an isometric view of one location for the artificial prosthesis of the present invention;

FIG. 2 is an isometric view of one embodiment of an intervertebral prosthesis of the present invention;

FIG. 3 is an exploded view of one embodiment of an intervertebral prosthesis of the present invention;

FIG. 4 is an inverse exploded view of the embodiment of an intervertebral prosthesis depicted in FIG. 3;

FIG. 5 is an isometric view of one disc of the stop, channel, pump and reservoir of the present invention;

FIG. 6 is an isometric view of the disc that is complementary to that depicted in FIG. 5; and

FIG. 7 is an isometric view that depicts the rotational movement of a spinal prosthesis in accordance with the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The discs of the present invention may be fabricated from generally biocompatible materials including, without limitation, titanium, surgical alloys, stainless steel, chrome-molybdenum alloy, cobalt chromium alloy, zirconium oxide ceramic, non-absorbable polymers and other anticipated biocompatible metallic, ceramic or polymeric materials and combinations thereof. The discs may be fabricated from different materials and may even be fabricated by using different materials within each disc. Certain materials may even be used to coat a portion or all of a disc, e.g., a titanium disc may be fused, welded, melted, coated, impregnated or infused along it's a portion or all of its periphery with a ceramic or a polymer. Alternatively, the fused, welded, melted, coated, impregnated or infused material may be on a portion or all of one surface but not another, or, may completely surround the disc.

The ion and liquid channel formed within the present invention may be formed on one or between two adjacent surfaces. Likewise, the reservoir and/or pump that is in fluid communication with the channel may be formed into or between adjacent surface. The reservoir for use with the present invention may serve a variety of purposes, from a reservoir for debris that results from the implantation and/or long term use of the prosthesis. The reservoir may also be used to delivery, immediately or long-term one or more active agents that act at or about the prosthesis to improve patient comfort, improve healing, decrease healing time, prevent rejection, prevent infection, prevent formation of scar tissue, decrease bleeding or combinations thereof. The exact active agents may be selected for each patient, patient condition, site of implantation and the like. The reservoir may be made accessible externally to remove and/or insert materials before, during or after implantation.

The one or more active agents that may be inserted, placed or replaced in the reservoir may include, e.g., one or more growth factors, one or more bone morphogenic protein isoforms, steroids, collagen, glucocorticoids, analgesics, anti-inflammatories and mixtures and combinations. The exact formulation or combination will depend on a variety of factors, however, the skilled surgeon may have one or more preferred combinations that may be used at different times. For example, the surgeon may start with an analgesic, anti-inflammatory combination for initial implantation. Depending on the recovery, the surgeon may next insert in the reservoir controlled-release bone morphogenic protein.

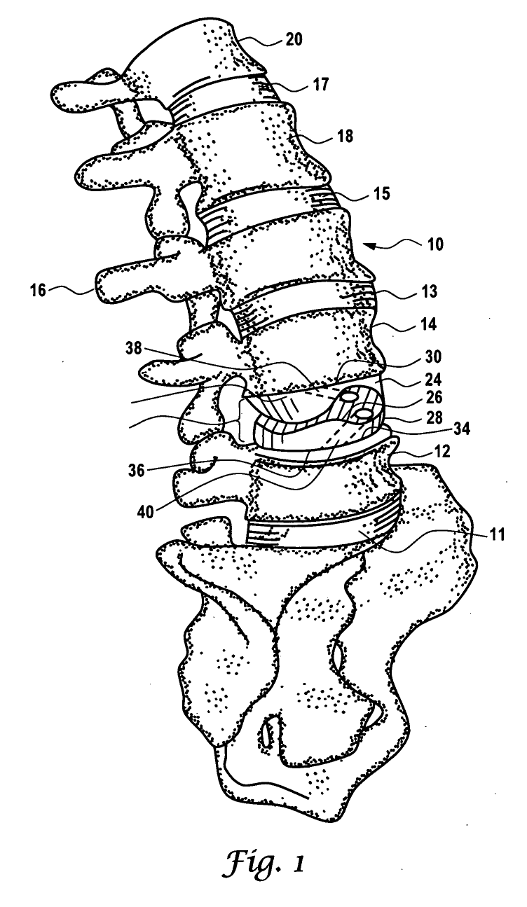

FIG. 1 depicts one specific embodiment of the present invention used as an artificial spinal disc replacement. As will be disclosed in detail herein, the present invention will have wide applicability to a number of different locations and for replacement of many different types of joints. The artificial spinal prosthesis depicted in FIG. 1 is a non-limiting example of the present invention. A partial isometric view of a human spinal column (10) is shown with various vertebrae (12, 14, 16, 18, 20) and various intervertebral spinal discs (11, 13, 15, 17). An artificial spinal disc (22) has a first plate (24), a second plate (26) and a third plate (28) interposed between a superior vertebra (14) and an inferior vertebra (12), respectively. The superior vertebra (14) has an inferior surface (30) depicted as planar, or flat, so as to optimize the substantially complementary upper surface (32) of the plate (24). The inferior vertebra (12) has a superior vertebral surface (34) that has a shape that is complementary to the inferior surface (36) of the plate (34). Although not shown, the musculature and other body tissues normally surround the spinal column around the artificial spinal disc (22) and the vertebrae (12, 14, 16, 18, 20) and intervertebral spinal discs (11, 13, 15, 17).

Each of the plates (24, 28) may include openings (38, 40) through which screws, nails, or other types of mechanical attachment facilitate the permanent localization of the artificial spinal disc (22) to adjacent vertebrae (in this example 12 and 14). A wide variety of mechanical attachments methods and hardware may be used ensure that the components of the artificial spinal disc (22) remain in place for the duration of their use. By way of example and without limitation, mechanical attachment may include a screw, nail, rivet, adhesive, wire, band, strap, and embodiments for porous coating of the prosthesis endplate screws or the combination and locking mechanisms to affix the screw to the artificial spinal disc (22). The openings (38, 40), if present, may be shaped as desire, e.g., the may be adapted to receive and retain screws, nails, rivets, spikes and other articles used to secure the discs (24, 28) to adjacent vertebrae without protruding from the surface of the discs (24, 28) to cause friction. For example, the openings (38, 40) may be countersunk or can have locking methods or mechanisms that stabilize a screw (not depicted) to the artificial spinal disc (22) prosthesis depicted in FIG. 1. Examples of methods and hardware for attaching a prosthesis to bone are well known in the art, e.g., U.S. Pat. No. 4,759,769, to Hedman, et al., U.S. Pat. No. 4,946,378, to Hirayama, et al., U.S. Pat. No. 4,997,432, to Keller, U.S. Pat. No. 5,002,576, to Fuhrmann, et al., U.S. Pat. No. 5,236,460, to Barber, U.S. Pat. No. 5,258,031, to Salib, et al., U.S. Pat. No. 5,306,308, to Gross, et al., U.S. Pat. No. 5,401,269, to Buttner-Janz, et al, U.S. Pat. No. 5,425,773, to Boyd, et al., and U.S. Pat. No. 5,782,832, to Larsen, et al., relevant portions incorporated herein by reference.

The artificial spinal disc (22) is shown in conjunction with a human spinal column 10, however, the artificial prosthesis of the present invention may be used for a wide variety of joints, including as: an artificial shoulder joint, an artificial hip joint, an artificial knee joint, an artificial elbow joint, an artificial ankle joint, an artificial wrist joint, an artificial carpo-metacarpal joint, an artificial metacarpo-phalangeal joint, an artificial interphalangeal joint and an artificial metatarso-phalangeal joint. Each of these joints will have unique flexion requirements and the present invention may be customized for each type of joint, and even for each individual patient by simply taking into account the following factors: (1) the size, surface and shape of the bones that form the joint; (2) the mechanical characteristics of the bones that form the joint and the prosthesis to be implanted; (3) the type of bone and bone structure to which the prosthesis will be attached; (4) to degree of rotation, flexion and limits that may want to be imposed on the movement of the joint; (5) the age of the patient; (6) other health requirements, health history and specific characteristics of the implantation site, etc. With these parameters established using any number of invasive and/or non-invasive procedures, the surgeon may even design a custom prosthesis having the basic characteristics disclosed herein to maximize patient health, minimize recovery time, minimize morbidity and provide a long-term solution to the needs of the patient.

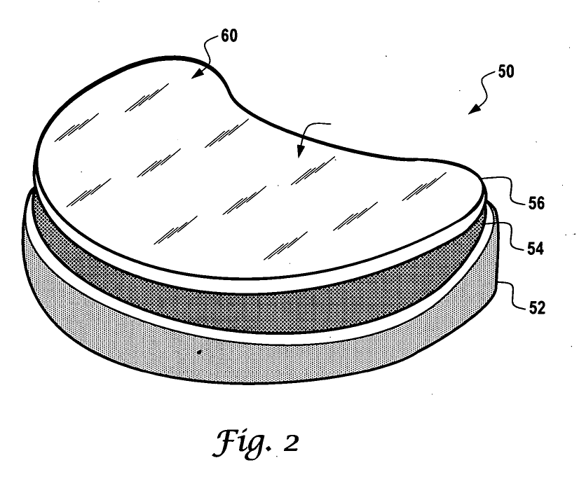

FIG. 2 is an isometric view of one embodiment of an intervertebral prosthesis (50) of the present invention. In this view, the intervertebral prosthesis (50) has three discs: inferior disc (52), medial disk (54), and superior disc (56), which are depicted as having a kidney-like shape. The upper surface (58) of the superior disc (56) is depicted as substantially planar, however, in certain embodiments of the present invention the upper surface (58) is contoured to match or complement its osseal counterpart. For example, in certain embodiments, the upper surface (58) of the superior disc (56) may include an internal opening or reservoir in which one or more active agents are provided. The opening or reservoir (or multiple openings and/or reservoirs) include one or more active agents, e.g., one or more growth factors, one or more bone morphogenic protein (BMP) isoforms, steroids, collagen, glucocorticoids, analgesics, anti-inflammatories and mixtures and combinations. The reservoir may be accessible externally such that matter may be removed (e.g., debris) or inserted (e.g., to change or replace the active agents). The one or more active agents may themselves be biocompatible, biodegradable and even provide for sustained release of the active agents.

The upper surface (58) of the superior disc (56) (and while not depicted also an analogous surface on the externally expose planar surface of the inferior disc (52)) may have the one or more openings that are apposite and provide a soft interface with the cancellous portion of the bone at the articular surface. The periphery or rim (60) of the superior disc (56) (and/or its inferior disc (52) counterpart) will provide the attachment point for the rim or load bearing portions of the bone, that is, the compact bone. The upper surface (28) (and/or its inferior disc (52) counterpart) may be generally, smooth, splined, flanged, spiked or beaded.

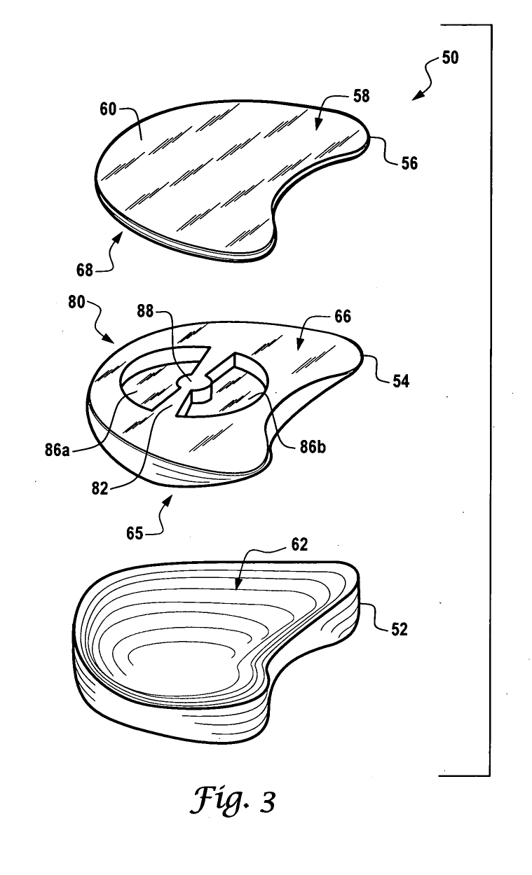

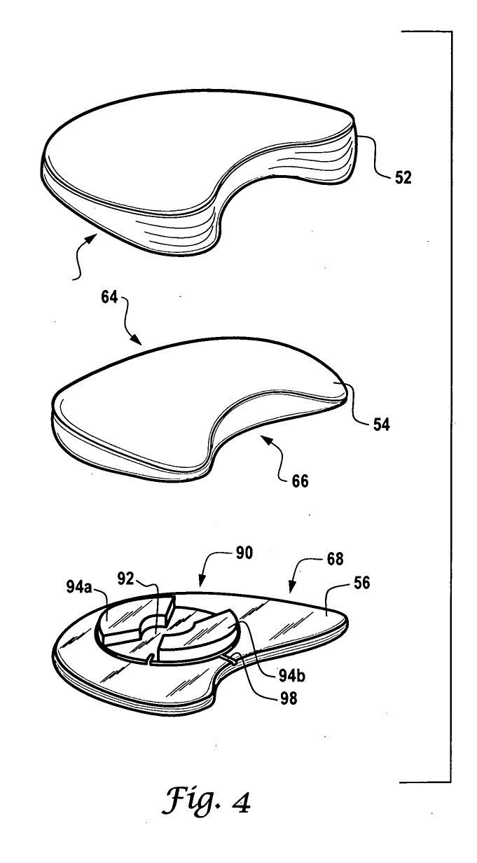

FIG. 3 is an exploded view of one embodiment of an intervertebral prosthesis (50) of the present invention that shows the interaction, shape and components that connect the three discs: inferior disc (52), medial disk (54), and superior disc (56) of the intervertebral prosthesis (50). FIG. 4 is an inverse exploded view of the embodiment of an intervertebral prosthesis depicted in FIG. 3. As shown in FIG. 3, the posterior surface (62) of inferior disk (52) is concave and is complementary to (see FIG. 4) the convex shape of inferior surface (64) of medial disk (54). As such, the complementary portions of inferior disc (52) and medial disc (54) provide the center of rotation and flexion of the intervertebral prosthesis (50). As discussed hereinabove, the exact location of the center of gravity may be varied to maximize the comfort and movement of the intervertebral prosthesis (50). For example, for the lower lumbar region, the center of gravity will generally be posterior to provide a generally anterior moment. Depending on the exact location of the intervertebral prosthesis (50) up and down the spine, the center of gravity may be changed to account for spin curvature, patient age or other conditions.

The posterior surface (66) of the medial disc (54) is depicted with an internal concave motion control mechanism (80) that includes a bilateral stop (82a, 82b), two concave openings (86), a central hub (88). The partially complementary to the internal motion control mechanism (80) is visible in FIG. 4 and is an internal convex motion control mechanism (90). The internal convex motion control mechanism (90) has a central hub mating portion (92) and two convex partially mating portions (94a, 94b). Partially visible in this view is channel (98). The central hub portions (88) and (92) generally limit the rotation of the medial disc (54) and superior disc (65) to pivot. The range of the pivot may be, e.g., up to 17 degrees from center in either direction. In some cases it may be preferable to limit the angle of pivot further, depending on the site of implantation, type of joint, age of the patient, etc.

One distinct advantage of the present invention is that the artificial prosthesis provides two separate ways to control, limit or encourage the type of motion between the discs in accordance with the needs of the implant location. For example, in certain embodiments, its possible to combine pivot control (between, e.g., the superior disc and the medial disc, as depicted) with a ball and socket joint, a hinge, a saddle, an ellipsoid a gliding or another pivot joint, and vice versa. By having two separate locations to control the range and type of motion, the present invention permits for the first time the use of a single device for a wide range of applications. For example, a finger joint would have very minimal pivotal motion and extension, but would require a wide range of flexion.

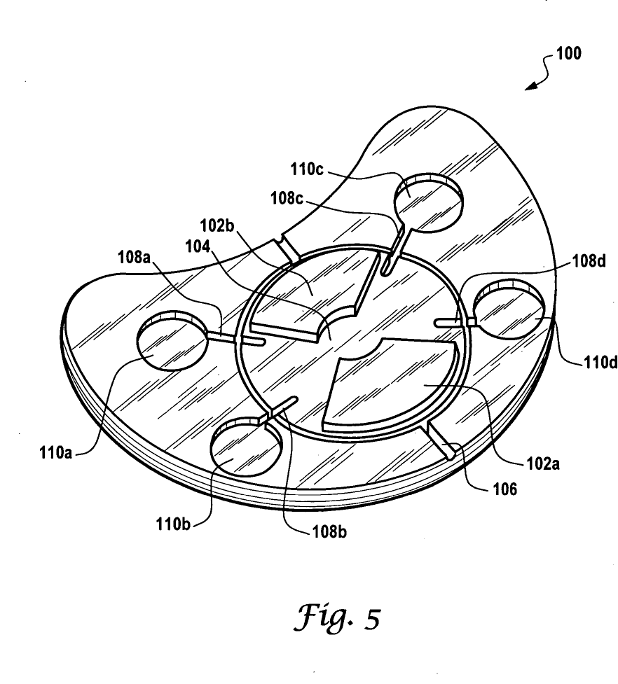

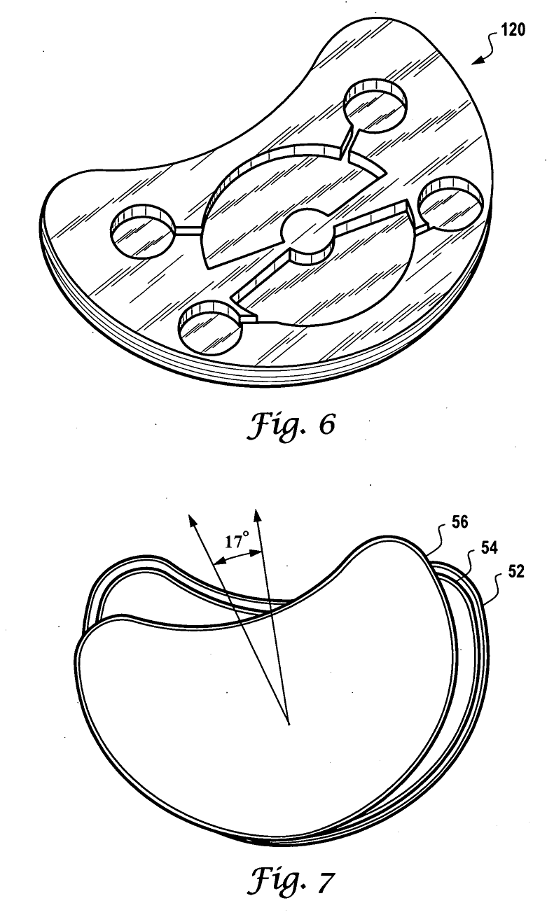

FIGS. 5 and 6 are isometric views of one disc (100) (and its complement (120)) that includes two convex stops (102a, 102b), a central hub opening (104), a channel (106) and reservoirs (108a, 108b, 108c, 108d) of the present invention. The opening (106) may be made externally accessible, e.g., to a syringe needle (not depicted) that permits the physician to add or subtract materials to the channel (106) and reservoirs (108a, 108b, 108c, 108d). Optional reservoirs (110a, 110b, 110c, 110d) are also depicted, which may be used to store debris caused by the friction of the parts or active agents. Depending on their shape and the motion of the joint, the reservoirs (118a-d and/or 110a-d, or combinations thereof) may act as one or more pumps that will permit or actively pump fluid through the channel (106). Importantly, it has been found that in locations with a low oxygen content, the intervertebral joints, the channel (106) reduces the “battery” effect that is cause by the superposition of charges in different portions of implants, in particular, metal implants that will tend to corrode and degrade due to the alignment of ions along a gradient that cause an ionic/electrical capacity to form. The corrosion causes degradation of the edges of the artificial prosthesis causing fraying of the edges, which leads to the build up of debris at the joint, which sharply decreases the longevity of the joint.

FIG. 7 is an isometric view that depicts the rotational movement of a spinal prosthesis in accordance with the present invention. As can be seen in this view, the pivot motion is limited in range to about 17 degrees in either direction. Using the present invention, a wide range of motion is possible and easy to control, from none to both anterior, posterior and from zero to 180 degrees in one, two or three dimensions.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. What is claimed is:

1. An artificial prosthesis comprising:
a first disc comprising a first disc periphery, a first disc superior surface and a first disc inferior surface that comprises a first disc mating portion;
a second disc comprising a second disc periphery, a second disc superior surface that comprises a second disc mating portion that is substantially complementary to the first disc mating portion and a convex inferior surface; and
a third disc comprising a concave superior surface that substantially complements the convex inferior surface of the second disc.

2. The prosthesis of claim 1, wherein the first, the second or both mating portions further comprise one or more grooves that extend to the first, second or both outer disc periphery, wherein the one or more grooves are in fluid communication at a site of implantation.

3. The prosthesis of claim 1, wherein the first, the second or both mating portions further comprise one or more grooves that extend to the first, second or both outer disc periphery, wherein the one or more grooves are in fluid communication at a site of implantation and permits ion transfer at, through and about the outer periphery of the first and second discs.

4. The prosthesis of claim 1, wherein the first, the second or both mating disc portions comprise one or more grooves that are shaped to create a mechanical fluid pump that causes the movement of fluid through the grooves.

5. The prosthesis of claim 1, wherein the first, the second or both mating disc portions comprise one or more grooves that are shaped to create a mechanical fluid pump that causes the movement of fluid through the grooves and one or more reservoirs adapted to collect particulate matter.

6. The prosthesis of claim 1, wherein the first, the second or both mating disc portions comprise one or more reservoirs for one or more active agents.

7. The prosthesis of claim 1, wherein the first, second or third disc comprise stainless steel, titanium alloy, cobalt alloy, anodized aluminum, a polymer, ceramics and composites materials or combinations thereof.

8. The prosthesis of claim 1, wherein the first and second mating disc portions limit motion up to about a 17 degree range of motion.

9. The prosthesis of claim 1, wherein the center of gravity of the convex surface of the third disc is off-center.

10. The prosthesis of claim 1, wherein the center of gravity of the convex surface of the third disk is selected based on an implantation site.

11. The prosthesis of claim 1, wherein the first, the second, the third or combinations thereof may further comprises an inferior surface comprising one or more reservoirs for storing one or more active agents.

12. The prosthesis of claim 1, wherein the first, the second, the third or combinations thereof may further comprise or form therewith one or more reservoirs for storing one or more active agents selected from one or more bone morphogenic proteins, antibiotics, steroids, glucocorticoids, analgesics, anti-inflammatories, mixtures and combinations thereof.

13. The prosthesis of claim 1, wherein the third disc further comprises an inferior surface comprising one or more sustained-release reservoirs for storing one or more active agents selected from one or more bone morphogenic proteins, antibiotics, steroids, analgesics, anti-inflammatories and mixtures or combinations thereof.

14. The prosthesis of claim 1, wherein the first, the third disc or both may further comprises an inferior surface comprising a periphery that complements and attaches to the load-bearing rim of a bone upon implantation.

15. The prosthesis of claim 1, wherein the third disc further comprises an inferior surface comprising one or more reservoirs for storing one or more active agents selected from one or more bone morphogenic proteins, antibiotics, steroids, analgesics, anti-inflammatories and mixtures or combinations thereof and an inferior surface comprising a periphery that complements and attaches to the load-bearing rim of a bone upon implantation.

16. The prosthesis of claim 1, wherein the prosthesis is an artificial shoulder joint, an artificial hip joint, an artificial knee joint, an artificial elbow joint, an artificial ankle joint, an artificial wrist joint, an artificial carpo-metacarpal joint, an artificial metacarpo-phalangeal joint, an artificial interphalangeal joint and an artificial metatarso-phalangeal joint.

17. The prosthesis of claim 1, wherein the first, the third or both discs comprise one or more holes therethrough for use in securing the first, the third or both discs to a bone.

18. The prosthesis of claim 1, wherein the first, the second, the third outer disk periphery, and combinations thereof, is chamfered.

19. The prosthesis of claim 1, wherein the first, the second, the third outer disk periphery, and combinations thereof, is kidney shaped.

20. The prosthesis of claim 1, wherein the first, the second, the third outer disk periphery, and combinations thereof, is coated.

21. A method for replacing a joint in a human comprising the steps of:
removing a joint from a human to provide an space defined by a first and a second bone;
inserting the prosthesis of claim 1 in the space; and
attaching the prosthesis to the first and second bones.

22. The method of claim 21, wherein said space is defined further as an intervertebral, a shoulder, a hip, a knee, an elbow, an ankle, a wrist, a carpo-metacarpal, a metacarpo-phalangeal, an interphalangeal and a metatarso-phalangeal space.

23. The method of claim 21, further comprising the step of adapting the mating surface between a bone at the site of implantation to be substantially complementary to the first disc and the third disc.

24. The method of claim 23, wherein the bone, the first disc, the third disc or combinations thereof are adapted to be substantially complementary.

25. A method for replacing an intervertebral joint in a human comprising the steps of:
removing a spinal disc from a human spine to provide an intervertebral space defined by a superior vertebra and an inferior vertebra;
inserting a prosthesis comprising: a first disc comprising a first disc periphery, a first disc superior surface and a first disc inferior surface that comprises a first disc mating portion;
a second disc comprising a second disc periphery, a second disc superior surface that comprises a second disc mating portion that is substantially complementary to the first disc mating portion and a convex inferior surface; and a third disc comprising a concave superior surface that substantially complements the convex inferior surface of the second disc, in the intervertebral space; and
attaching the prosthesis to the superior and inferior vertebrae.
